(19) United States
(12) Patent Application Publication
(10) Pub. No.: US 2010/0015714 A1
(43) Pub. Date: Jan. 21, 2010

(54) MEASUREMENT OF SOIL POLLUTION

(76) Inventors: Selwayan Saini, Bedfordshire (GB); John Stephen Setford, Bedfordshire (GB); Julian Lawrence Ritchie, Bedfordshire (GB); Vincent Paul Knight, Bedfordshire (GB); Markus Michael Malecha, Bedfordshire (GB)

Correspondence Address: STITES & HARBISON PLLC, 1199 NORTH FAIRFAX STREET, SUITE 900, ALEXANDRIA, VA, 22314, US

(21) Appl. No.: 11/720,694

(22) PCT Filed: Dec. 5, 2005
(86) PCT No.: PCT/GB05/04652

§ 371 Date: Feb. 27, 2009

(30) Foreign Application Priority Data
Dec. 4, 2004 (GB) 0426696.1

(51) Int. Cl. G01N 33/26

(52) U.S. Cl. 436/25

(57) ABSTRACT

A soil sample of fixed volume is mixed with a drying agent (MgSO₄) and then acetone. The liquid is filtered off and a sample is applied to the sensing surface of an attenuated total reflectance (ATR) device in an IR spectrometer. After evaporation of the acetone, absorption is measured in a C-H stretch region (e.g. 2950 cm⁻¹) to provide a value indicative of the amount of oil in the sample.

### TECHNICAL FIELD

The present invention relates to a method for the measurement of soil pollution.

Current and planned legislation is forcing industry to comply with increasingly stringent pollution consent levels. The European Union has framed a statute enshrining the dictate ‘the polluter pays’, while the US EPA has developed stringent regulations with significant penalties for industrial polluters. Organisations shown to be in non-adherence to a given environmental protection directive will not only be liable to prosecution, but will also be responsible for regenerating the polluted area to an acceptable state. Industry has reacted by adopting environmental monitoring practices. Typically soil, water or air samples are taken from the area of concern and are shipped to a remote laboratory for analysis. However, laboratory-based analytical techniques tend to be expensive to maintain, requiring complex and costly instrumentation, frequent recalibration and highly trained personnel. Consequently, there has been a clear identifiable need for chemical measurement tools that can be used on location to provide accurate site-wide, low-level contamination measurement for land redevelopment. Such tools are particularly attractive to commercial operators and legislators as they provide immediate information on the state of contamination.

Of all the pollution incidents, fuel and oil pollution are the greatest, responsible for 90% of all hazardous organic contamination across Europe. Contamination may be caused by, for example, underground and overground storage tanks, oil and electrical pipelines, filling stations, site chemical storage, and users of hydraulic oils and vegetable oils. The UK Environment Agency estimates that ⅓ of petrol stations have a pollution problem and the US EPA expects 75% of all underground oil storage facilities to fail within the next decade. This is a huge problem affecting ground and drinking water quality globally. Indeed there are approximately 15 million sites in the developed world that are or may become contaminated by oils and require measurement in order to target remediation.

### BACKGROUND ART

Current techniques involving taking samples to laboratories for analysis by sophisticated techniques using large, expensive, complex equipment such as GC-FID (gas chromatography with flame ionisation detection).

Currently there is no rapid, portable ergonomically simple extraction and measurement system on the market capable of producing such accurate measurements at moderate cost. It would be advantageous to have a device suitable for use in, for example, one or more of: portable oil leak detection along cable or pipeline runs, oil spill movement tracking (this is necessary once pollution occurs in an aquifer system); land valuation assessment; remediation monitoring; housing development; anywhere where fast results are needed. In the British national power delivery sector (not local delivery) alone leakage from underground power cables can cost the industry up to ±250,000 per day in lost business and loss of network security, with the only method of leak detection available involving exploratory digging until the leak is located. The fuel leak detection market is worth in excess of ±10 billion per annum, much of that in lost diesel.

### DISCLOSURE OF INVENTION

According to the present invention there is provided a method of quantifying oil contamination of soil comprising:

(i) taking a sample of soil having a predetermined volume;

(ii) mixing the soil sample with a drying agent;

(iii) adding acetone to the soil sample;

(iv) mixing the soil sample/drying agent/acetone;

(v) filtering to obtain a liquid phase;

(vi) applying a sample of the liquid phase of predetermined volume to an attenuated total reflectance (“ATR”) crystal surface of an infra-red (“IR”) spectrometer

(vii) allowing acetone to evaporate from the liquid phase sample on the ATR crystal surface;

(vii) using the spectrometer to determine IR spectrographic data relating to the sample; and

(ix) obtaining data indicative of the oil content of the soil sample from said IR data.

The ATR crystal is preferably of zinc selenide. Other possibilities include germanium, zirconia and diamond.

This method can be used for the rapid on-site measurement of oil and fuel contamination in soils. The combined OMD and extraction mechanism operates by measuring the absorption of infrared light due to C—H bonds present in oil extracted from soil and deposited on an attenuated total reflectance (ATR) crystal surface, after evaporation of the volatile solvent evaporation phase. The extraction of the soil sample uses, in the same step, an oil extraction AND drying arrangement, suitable for all “normal” soil water concentrations up to 30%, negating the need for spectral correction due to water content of sample, leaving an extract ready for filtering and depositing on the sensor surface.

Solvents other than acetone could be used, particularly other volatile organic solvents such as other ketones, alcohols, esters, ethers and hydrocarbons.

### BRIEF DESCRIPTION OF DRAWINGS

FIG. 1 is a schematic view of apparatus for carrying out an embodiment of the invention.

FIG. 2 is a diagram showing unprocessed output data;

FIG. 3 is a diagram showing processed data;

FIG. 4 is a calibration curve;

FIG. 5 is a block diagram of the electronic components;

FIG. 6 shows a calibration curve.

FIG. 7 displays test data for five soils, sampled on two different days.

FIG. 8 displays test data for the same five soils determined by a method embodying the invention and by two other methods.

### BEST MODE FOR CARRYING OUT THE INVENTION

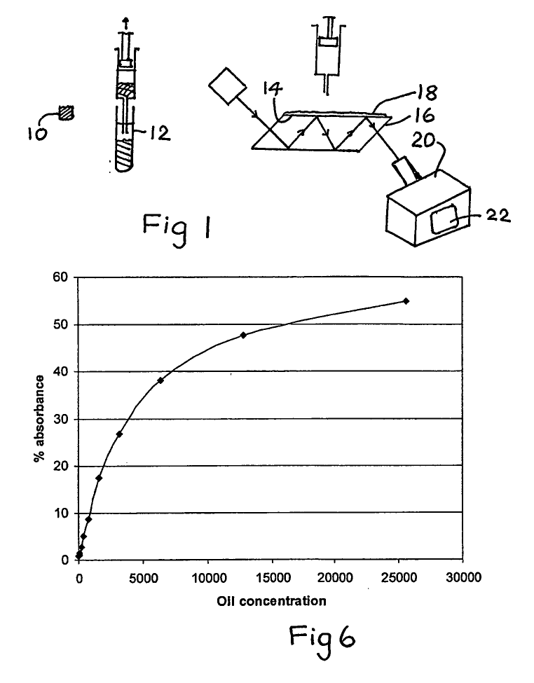

As shown in FIG. 1, a sampling vessel 10 is used to collect a known volume of soil (e.g. 5 ml). Preferably some care is taken to avoid macroscopic vegetable matter such as roots and other plant parts, and stones. The soil sample is placed in a larger vessel, e.g. a 50 ml centrifuge tube 12. An aliquot of anhydrous magnesium sulphate (e.g. 2 g) and an aliquot of acetone (HPLC grade, e.g. 10 ml) are added, and the mixture is briefly stirred and then shaken, e.g. for 2 minutes. The acetone phase is separated, e.g. by filtration using filter paper or a membrane syringe. A measured volume (e.g. 100 μl) is applied to the sensor surface 14 of a zinc selenide ATR crystal device 16 of an IR spectrometer. The ATR crystal is a Specac HATR trough top plate GS 111 66 (www.specac.com). The acetone is allowed to evaporate, e.g. for 2 minutes, so that a film 18 of oils present in the soil sample is deposited on the sensor surface. The spectrometer is operated.

The optimal way of measuring MIR light throughput is by using a changing, or oscillating light signal, so that differences between transmission at maximum source output and minimum source output can be quantified. This negates any system offset and makes unnecessary measurement of fine, or drifting differences between absolute signal values. Two channels—a signal channel and a reference channel are used so that any change in operating conditions, e.g. due to external temperature, or state of battery charge, which may affect absolute signal values, will minimally affect values based on signal differences or reciprocal values.

The source should be low thermal mass heater which is preferably capable of electronic modulation (or the output could be mechanically chopped). We used a high temperature thin film element with parabolic back-reflector to minimise light wastage. It is preferably pulsed at five Hertz. (Other frequencies up to 15 Hz, e.g. 8 Hz may be used). It reaches a maximum colour temperature of approximately 1000° C. for a fraction of a second whilst pulse power is applied. In between pulses it cools off to near ambient. At peak power it uses 1 W. This device has very significant light output at the C-H absorption energy of 2950 cm⁻¹, imperative for the sensitive measurement of hydrocarbon absorption. The emitter of choice is a windowless IR55 unit with parabolic reflector from Scitec (Redruth, Cornwall, GB, www.scitec.uk.com).

The emitter and detector are placed at the ATR crystal faces to get maximum throughout of light. Six reflections at the sensing surface gives maximum opportunity for evanescent wave absorption by the C—H bonds in the sample.

The detector of choice is a pyroelectric detector. This device is designed for broad-band IR measurement. The hot element inside the component is made of a highly ferroelectric material which, when maintained below its Curie temperature, exhibits large spontaneous electrical polarisation. If the temperature of the filament material is altered, for example, by absorption of incident radiation, the polarisation changes, which is measured as a capacitance change, monitored using transient detection electronics. This process in independent of the wavelength of the incident radiation and hence pyroelectric sensors have a flat response over a very wide spectral range. The specificity of the device is modified by two bandpass filters, allowing only radiation of the correct wavelength to interact with the pyroelectric material. The component of choice is a Pyromid LMM 242D made by Infratec (available from Lasercomponents (UK) Ltd, details www.infratec.de). This is a dual channel pyroelectric detector with inbuilt amplification, and specificity at 3400 nm (2900 cm-1), with a reference channel at 3950 nm (2531 cm-1), both channels created by the use of notch filters over the relevant detector filament. The reference channel is made available so that a ratiometric measurement can be made using the same source, thus accounting for intensity variation as a function of instantaneous source power. This has the benefit of making the device less prone to electronics variations as a function of power supply or ambient thermal fluctuation.

In operation, a high-power collimated beam of IR radiation is passed into the ATR crystal 16 where it undergoes internal reflection, including reflections off the sensor surface 14, before leaving the crystal and passing to the detector 20.

The electrical driving impulse for the emitter is specially shaped for fast optical output rise-time. An ATR of zinc selenide is suitable since this material is compatible with the extraction protocol solvents.

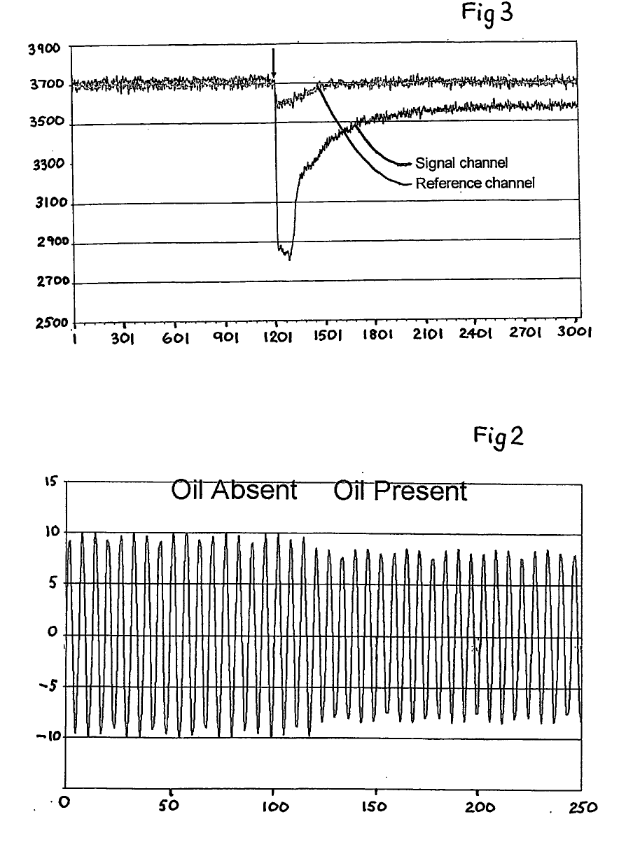

Data processing is a vital post-collection function for accurate and repeatable work to be done. The actual measurements that are made in the device are nano-volt changes in the detector voltage output due to the capacitance change caused by variation in the intensity of light passed through the ATR crystal as the light emitter is pulsed on then off, five times per second. The difference in the light throughput between on and off stages is the signal collected. There are two channels in the detector of this device, both collecting light from the emitter passing through the crystal, each operating at a particular wavelength. The first channel measures the throughput of light at the peak wavelength of absorption of hydrocarbon bonds (wavelength 3.4 μm or energy 2950 cm⁻¹). The second channel measures throughput of light at a wavelength where very few compounds absorb, and this is the reference channel (wavelength approximately 3 μm). It is two-channel so that division of signal channel signal by reference channel signal compensates for external temperature variation, power-supply fluctuation or natural deterioration of any of the electrical parts over their useful lifespan, such as the light source. FIG. 2 shows graphically the electronic signal received from the pyroelectric detector, before processing and display. It is an AC signal with intensity on the Y-axis, and time (in 25^{ths} of a second) on the x-axis.

The data presented show diagrammatically the signal output from the detector in the presence and absence of oil. The change is so small that it is affected very strongly by noise, hence algorithms have been designed to minimise these effects by finding correlation over many cycles, compensating for a) high frequency “within one cycle” noise, b) variation of peak height over a period of seconds c) variation over minutes and hours, or instrument drift, d) drift over the lifespan of the components (measured in years).

Peaks are mapped with twenty data points per peak (limiting high frequency noise), and their height is measured as distance away from the average depth of the troughs to either side (compensating for minutes drift). A moving average of these values is taken prior to the addition of sample (data points are collected all the time) and for 30 s after the carrier solvent has evaporated. The difference between these two levels is then mapped to the in-built calibration statistic and the most recent calibration curve data. Several tests were made regarding absolute performance of the device. Actual signal data for the addition of oil in acetone at 200 ppm are shown in FIG. 3.

Once the contributions of all the peaks are averaged, the signal channel and reference channel are displayed as continuous DC signals. The difference between the height of the signal channel before and after sample addition (large central dip) is related to the amount of oil added to the ATR surface. Intensity is displayed on the y-axis with time (5^{ths} of a second) on the x-axis

The y-axis expresses counts with no units specified (it is a reciprocal measurement). The oil in acetone (200 ppm) was added after four minutes background collection time. The response it induces in the sensor is immediate and very large because of the enormous amount of acetone present in the sample, which strongly affects the signal channel, and even causes change in the reference channel As the acetone evaporates both signals tend to a resting level. The reference channel returns to the level it was before the addition. With the signal channel the final level is proportional to the amount of oil left on the sensor surface once the acetone has evaporated. The software logs the data and detects this large change in absorbance due to the addition of the acetone. It then calculates the initial signal level prior to acetone addition. It then waits two minutes for the acetone to evaporate and calculates the final signal level. The comparison is made between this absorbance and the calibration absorbance to calculate the amount of oil present.

It is important to collect data for a sufficiently large measurement period that a good average signal is collected, so minimising the noise component of the signal. Equally it is important that the measurement time is not increased beyond a reasonably short period, to avoid data loss through too lengthy a measurement. Data collection times were therefore kept to a minimum, totalling one minute per measurement, with four minutes total time allowed for evaporation of solvent (two minutes prior to and two minutes after solvent addition). It is important to note that, should a more precise reading for soil contamination be needed, it is possible to increase the measurement time. This would have two effects. Firstly it would allow averaging to occur over more cycles, reducing uncertainty. Secondly it would allow greater stabilisation of the device following the perturbation applied by adding the sample. Allowing longer for this decreases the uncertainty; however this would increase measurement time, and as one of the goals of this project is to reduce measurement time as much as possible, a compromise has been reached between precision and time for measurement to take place.

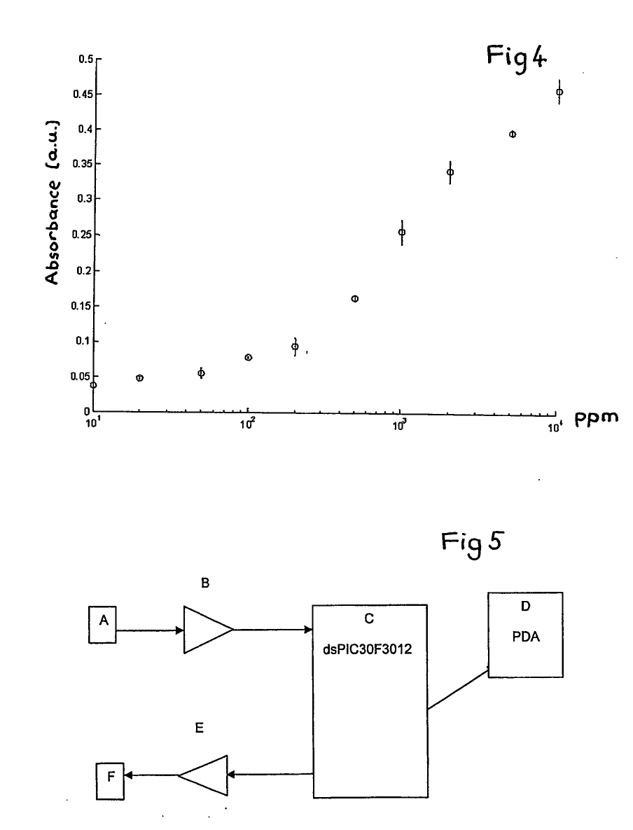

An optimised calibration curve is shown below in FIG. 4. This includes data from only one machine setting: the collection of peak heights for 30 s following a two minute evaporation period. This is a compromise between measurement precision and time taken, since it is a requirement of the specification that sampling time be reduced as much as possible.

FIG. 5 shows a block diagram of the electronics.

The dual channel detector (A) sends low level signals (+/−0.1 V) to the offset voltage amplifier (B) which scales the voltage from 0 to 5V for the Microchip dsPIC30F3012 (C). This contains a 12 bit A to D converter running at 2 kHz sample rate. An algorithm detects all peaks and troughs and measures trough depth from an average of the height of each of the surrounding peaks to help combat longer-term drift. The chip contains a DSP (digital signal processing) algorithm which acts as a bandpass filter allowing frequencies between 6 and 37 Hz to pass, eliminating mains noise (50 Hz) and longer-term drift. It is a 247 point finite impulse response filter, optimised for 8 Hz. The chip also outputs a 8 Hz pulse width modulated TTL signal which is amplified and current-boosted by amplifier circuit E, to drive the IR55 emitter F. The signal operates the emitter most efficiently at a mark-space ratio of 65%. The RS232 link is used to communicate data to the PDA (D) for data display. (Note: In this embodiment the emitter repetition rate has been increased from 5 Hz to 8 Hz to decrease measurement time, though a simple change in code would drop this once more to 5 Hz, and the bandpass would change slightly also.)

The device measures the concentration of extractable oils automatically It is vitally important that soil is taken by volume rather than mass, since the (unknown) water content strongly affects density and therefore the amount of soil in a sample taken by mass. The soil is pre-mixed with the drying agent to optimize water uptake prior to acetone addition. The ability of magnesium sulphate (anhydrous) to dry solvents has been demonstrated elsewhere. Two minutes shaking allows strong permeation of acetone into the soil, dispersing large clumps of compacted soil. Following deposition onto the sensing surface, most evaporation is completed after only 60 seconds, however 2 minutes is given to ensure complete loss of the volatile component. Measurement is complete after a further 30 seconds and is displayed on-screen.

The system offers the following advantages:
1) A sub 10-minute field method for quantitative extraction and measurement of oil contamination from soils
2) Combined completely portable extraction step and quantitative measurement system, requiring minimal operational training
3) An extraction step eliminating the effect of naturally occurring water in the sample which would otherwise adversely affect the IR measurement.
4) A quantitative evaporative oil deposition step following extraction using a non-chlorinated, low toxicity volatile solvent
5) Two channel, low cost, pyroelectric detection system with onboard AC signal deconvolution algorithms designed for the shaped-pulse IR emitter coupled with the rate-of-change detector
6) Inherent insensitivity to calibration drift owing to ratiometric measurement
7) The measurement is largely automated and the extraction is prompted by PDA (personal digital assistant, e.g. an HP Ipaq).

### Device Response

The device was calibrated between 0 ppm and 25600 ppm (v/v) using standards. Application of standards following calibration showed that the standard deviation for each point was less than 4% total absorbance. This gives an idea of the precision of the instrument. No calibration drift was observed when standards were measured over a period of months of use. FIG. 6 shows an example of the type of calibration curve used for measurement. It is a graph of % absorbance, measured by the detector, vs the oil content in ppm of standard samples. Each bar represents 5 readings.

### Validation of the Device

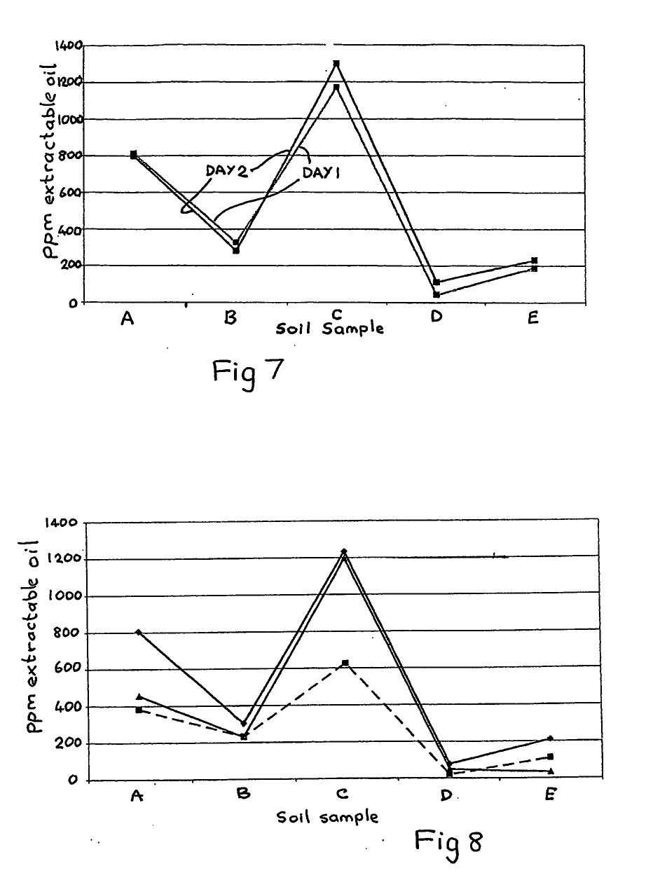

Analysis of five test soils brought from a site contaminated with electrical insulation oil, was performed on two separate days, with the results shown in FIG. 7. The precision of the extraction method and device is clear, with the majority of the pairs of results (for analysis of the same samples on different days) being within 20% of one another.

The results from blind measurement of the five soils tested using the device were compared with results produced by an independent laboratory, using two different techniques: extraction with perchloroethylene and measurement by benchtop FTIR, and extraction using EPA methods and measurement by GC/FID. The results are displayed in FIG. 8, wherein the top line (diamonds) is our results, the second line (triangles) shows the results using FI-IR and the bottom line (broken line, squares) shows the results using GC-FID. It is to be expected that there will be some differences between the two infrared measurement methods (ours and the FI-IR results) since the external laboratory uses a different extraction solvent for the soils. Indeed that used in the external laboratory is a much less environmentally friendly chlorinated solvent for extraction and measurement. The method developed for use with the new device specifically aimed to avoid the use of such hazardous materials. The device also fared well in comparison to analysis using the ‘gold standard’ EPA series of methods for extraction and analysis of Total Petroleum Hydrocarbons by GC/FID. The results by GC/FID are expected to be much less than those by IR since the GC/FID method only takes into account substances eluting between two time check points on a chromatogram representing a C10 and a C40 molecule, which is only a subset of the whole extractable material. Although there is more information available using the GC/FID method, it requires a laboratory fully equipped with expensive equipment with operation and analysis by trained personnel. The entire process may take over an hour per sample. The method suggested here produces a result within six minutes, has low initial and operational costs and is operable following minimal training.

1. A method of quantifying oil contamination of soil comprising:
(i) taking a sample of soil having a predetermined volume;
(ii) mixing the soil sample with a drying agent;
(iii) adding acetone to the soil sample;
(iv) mixing the soil sample/drying agent/acetone;
(v) filtering to obtain a liquid phase;
(vi) applying a sample of the liquid phase of predetermined volume to an attenuated total reflectance (“ATR”) crystal surface of an infra-red (“IR”) spectrometer
(vii) allowing acetone to evaporate from the liquid phase sample on the ATR crystal surface;
(vii) using the spectrometer to determine IR spectrographic data relating to the sample; and
(ix) obtaining data indicative of the oil content of the soil sample from said IR data.

2. A method according to claim 1 wherein the drying agent is magnesium sulphate.

3. A method according to claim 1 wherein the ATR crystal is zinc selenide.

4. A method according to claim 1 wherein the spectrometer measures absorption in the C-H stretch region to obtain a signal value.

5. The method of claim 4 wherein the spectrometer measures absorption at 2950 cm⁻¹.

6. The method of claim 4 wherein the spectrometer also measures absorption in a reference region to obtain a reference value.

7. The method of claim 6 wherein the reference value is measured at 2530 cm⁻¹.

8. The method of claim 6 wherein the ratio of signal value: reference value is periodically computed automatically.
